(19) United States
(12) Patent Application Publication
(10) Pub. No.: US 2011/0195921 A1
(43) Pub. Date: Aug. 11, 2011

(54) ELIMINATION OF A CONTAMINATING NON-HUMAN SIALIC ACID BY METABOLIC COMPETITION

(76) Inventors: Ajit Varki, La Jolla, CA (US); Sandra Diaz, San diego, CA (US); Rachel Taylor, Menlo park, CA (US)

(21) Appl. No.: 13/062,069

(22) PCT Filed: Sep. 9, 2009
(86) PCT No.: PCT/US2009/056361

§ 371 Date: Apr. 18, 2011

Related U.S. Application Data
(60) Provisional application No. 61/095,414, filed on Sep. 9, 2008.

(51) Int. Cl. A61K 31/70; C12N 5/02

(52) U.S. Cl. 514/42; 435/383; 435/375

(57) ABSTRACT

The disclosure provides a method of reducing or eliminating Neu5Gc in a cell culture or in a human subject. The method includes flooding the system with the human sialic acid i\7-acetylneuraminic acid (Neu5Ac) in glycosidically-bound or free form, or its precursor N-acetylmannosamine (ManNAc) in an amount sufficient to metabolically compete out the Neu5Gc, either as it enters the cells for the first time or when it recycles from break-down of preexisting cellular molecules. Additionally, Neu5Ac feeding results in reduction of Neu5Gc expression even in some animal cells capable of Neu5Gc production.

### CROSS REFERENCE TO RELATED APPLICATIONS

This application claims priority under 35 U.S.C. §119 from Provisional Application Ser. No. 61/095,414, filed Sep. 9, 2008, the disclosure of which is incorporated herein by reference.

### STATEMENT AS TO FEDERALLY SPONSORED RESEARCH

The U.S. Government has certain rights in this invention pursuant to Grant No. GM032373 awarded by the National Institutes of Health.

### TECHNICAL FIELD

This application is in the field of sialic acid chemistry, metabolism, and antigenicity. More particularly, this application relates to N-glycolylneuraminic acid (Neu5Gc) a non-human sialic acid that is immunogenic in humans, the production of Neu5Gc-free mammalian products for laboratory and human use, and the elimination of Neu5Gc from the human body.

### BACKGROUND

All cells are covered with a dense and complex array of sugar chains. Sialic acids (Sias) are a family of nine-carbon sugars that are typically present at the outer-most units of these chains. By virtue of their terminal position, sialic acids act as binding sites for many exogenous and endogenous receptors such as the Influenza viruses and the Siglec family of endogenous proteins. Such sugars are thus useful drug targets for the prevention and treatment of infection. They are also involved in various biological and pathological processes such as neuronal plasticity and cancer metastasis. In many of these instances, the precise structures of the sialic acid and the residues it is attached to play critical roles.

Cytidine monophosphate-N-acetylneuraminic acid hydroxylase (CMAH) converts the sialic acid N-acetylneuraminic acid (Neu5Ac) to N-glycolylneuraminic acid (Neu5Gc). In non-human mammals, Neu5Gc is recognized by a number of endogenous binding proteins, as well as by pathogenic organisms such as bacteria and viruses. Humans are unable to produce endogenous Neu5Gc because of an evolutionary inactivating mutation in their CMAH gene.

Furthermore, Neu5Gc is known to be immunogenic in humans. Such immunogenicity is believed to play a role in the immune response observed in humans that come into contact with mammalian products, such as cosmetics, food, mammalian cells and cell products, as well as therapeutic agents derived from non-human mammals or exposed to non-human mammalian products. Attempts have been undertaken to try to diminish the Neu5Gc content of recombinantly produced human glycoproteins in cell lines by altering the cell lines using RNAi to suppress expression of the CMAH gene (Chenu S., et al., Biochim. Biophys. Acta., 1622(2):133-144, 2003).

### SUMMARY

The non-human sialic acid N-glycolylneuraminic acid (Neu5Gc) contaminates both biotherapeutic products and the human body by becoming metabolically incorporated into cultured cells and into human tissues, respectively. In the first case the contamination arises from animal derived components in the culture medium and/or the animal cell lines used, and in the second case, from dietary intake from foods such as red meats. The methods of the disclosure eliminate or reduce the amount of Neu5Gc in human subject, biotherapeutics or cell lines. The method comprises flooding the system with the human sialic acid N-acetylneuraminic acid (Neu5Ac), derivatives, analogues or precursor thereof (e.g., N-acetylmannosamine (ManNAc)). The Neu5Ac used can also be bound in monomeric form to other molecules, or in a polymeric form (polysialic acid). In these cases, the bound Neu5Ac would be released by cellular processes and then act in a manner similar to the bound form. Both Neu5Ac and Neu5Gc compete for activation by the enzyme CMP-Sia Synthetase. Thus, and excess of Neu5Ac from any source provides a simple and effective method for reducing or eliminating the Neu5Gc burden by metabolically competing out the Neu5Gc, either as it enters the cells for the first time and/or when it recycles from breakdown of preexisting cellular molecules. In addition, the disclosure provides methods for reducing the production of Neu5Gc in CMAH-positive animal cells comprising flooding the system with a large bolus or increasing concentrations of Neu5Ac, which suppresses endogenous production of Neu5Gc.

The disclosure provides a method comprising, culturing a cell line with an effective amount or increasing amounts of N-acetylneuraminic acid (Neu5Ac), derivatives, analogues or precursors thereof in a sufficient amount for a sufficient period of time to eliminate or substantially reduce Neu5Gc present in a cell or the cell line or a product produced by the cell or cell line. In one embodiment, the cell line comprises a mammalian cell line used for therapeutic protein production. In another embodiment, the cell line comprises an hESC, an induced stem cell, an embryoid body cell or any other cell that is meant for use in therapeutic applications in humans or for use in the production of a therapeutic product.

The disclosure provides a method of reducing the amount of Neu5Gc in a biotherapeutic product comprising: culturing a mammalian cell culture under conditions for production of a biotherapeutic product in a medium comprising Neu5Ac.

The disclosure further provides a method of preparing a Neu5Gc-containing tissue for transplantation comprising exposing the tissue to media lacking Neu5Gc and comprising adequate amounts of N-acetylneuraminic acid (Neu5Ac) or its precursor N-acetylmannosamine (ManNAc).

The methods and compositions to eliminate Neu5Gc are applicable to human, as Neu5Ac (derivatives or analogues or precursors thereof) are small molecules that can diffuse to all compartments in the body and thus affect all cells in the body. The disclosure thus provides a method of treating an inflammatory disease or cancer comprising administering to a subject increasing amounts of N-acetylneuraminic acid (Neu5Ac) in bound or free form, or its precursor N-acetylmannosamine (ManNAc), until the level of Neu5Gc is reduced or removed from the subject. For example, the disclosure demonstrates that mice exposed to 1 mM Neu5Ac in the drinking water over 6 months developed no obvious toxicities, indicating that the process is safe for prolonged use in humans.

The disclosure also provides a food, beverage or cell media product having elevated Neu5Ac or its precursor compared to the level of Neu5Gc. In one embodiment, the food, beverage or cell media comprising from about 0.1 mM to 20 mM or more of Neu5Ac, in bound or free form.

The disclosure also provides a method of reducing production of Neu5Gc in an animal comprising a Neu5Gc enzymatic pathway, the method comprising administering to the animal an effective amount of Neu5Ac, derivative, analogue, or precursor wherein the effective amount reduces or eliminates that production of Neu5Gc in the animal.

In all of the above methods for animal use (including human use), the Neu5Ac can be in the form of a dietary supplement, as part of natural dietary product comprising high levels of Neu5Ac, synthetic Neu5Ac, monomeric or polymeric forms, bound or unbound forms of Neu5Ac. For culture methods, the Neu5Ac may be added to the media in bound, unbound form from synthetic or other production methods. The Neu5Ac may be in a substantially purified form for administration or dietary consumption.

The details of one or more embodiments of the disclosure are set forth in the accompanying drawings and the description below. Other features, objects, and advantages of the disclosure will be apparent from the description and drawings, and from the claims.

### BRIEF DESCRIPTION OF THE DRAWINGS

FIG. 1 show graphs of Neu5Gc content in two cell lines with and without Neu5Ac addition. Stably transfected CHO-KI cells expressing recombinant soluble Siglec 9-Fc or Siglec 13-Fc protein were grown in the absence or presence of 5 mM Neu5Ac (Ac). CHOSig9 cells were grown to 70% confluency in MEM alpha with 10% FCS and 80 μM MTX. CHOSig13 cells were grown to 70% confluency in MEM alpha with 10% FCS and 1.2 mg/ml G418. The cells were first rinsed twice with PBS before adding Optimem media with 2% Low IgG FCS and 5 mM Neu5Ac for three days, the media was collected on the third day (day 3), fresh media containing Neu5Ac was added and collected 2 days later (day 5), fresh media containing Neu5Ac was again added and collected on day 7. The individually collected media was centrifuged to remove cell debris, adjusted to 5 mM Tris-HCl pH 8 and incubated with 250 μl of Protein-A Sepharose for 3 days at 4° C. to bind the chimeric protein. The resin for each time point and cell type was washed with PBS and eluted with 0.1M Glycine pH 3. After neutralization to pH 7 with 1M Tris-HCl pH 8, the eluted protein was concentrated using a Millipore Ultrafree-15 30K cut-off device. The protein concentration was determined using Pierce BCA protein assay kit. An aliquot of the purified protein was hydrolyzed with 2M Acetic acid for 3 hrs at 80° C. to release the sialic acids followed by derivatization with DMB reagent. HPLC analysis was performed by reverse phase chromatography on a C18 column in the isocratic mode with 85% water, 8% acetonitrile and 7% methanol at 0.9 ml/min and the fluorescence detected. The area under each peak was obtained and the percent of Neu5Gc in each sample was determined relative to Neu5Ac. The results show that the amount of Neu5Gc attached to both proteins was markedly reduced by the addition of Neu5Ac. This provides an an approach to reduce Neu5Gc contamination in biotherapeutic products produced in CHO cells

FIG. 2A-E shows an approach to reduce Neu5Gc contamination in biotherapeutic products made in human cells that have accumulated Neu5Gc from the environment. (A-B) Feeding of human 293T cells with free Neu5Ac reduces pre-existing Neu5Gc content in glycoconjugates. Human 293T Cells were grown in DME+10% FCS, in the presence of 5 mM Neu5Gc for 3 days to load the cells with Neu5Gc. The cells were then washed with PBS, split into two identical cultures, and one had 5 mM Neu5Ac added while the other did not. Cells were then cultured for the several days as shown on the graph, harvested, and the Neu5Gc and Neu5Ac content of both the ethanol soluble low-molecular weight fraction (A) and ethanol precipitable proteins (B) analyzed by HPLC. The % Neu5Gc shown is the amount of Neu5Gc relative to the total sialic acids. It can be seen that the presence of Neu5Ac in the medium markedly accelerated the rate of Neu5Gc elimination from the cells. (C-E) Feeding of CHO cells with free Neu5Ac reduced Neu5Gc in the whole cell membranes and in secreted glycoproteins of an animal cell line with endogenous CMAH. Stably transfected CHO-KI cells expressing a recombinant soluble IgG-Fc fusion protein were grown in the absence or presence of 5 mM Neu5Ac (Ac). The cells were first rinsed twice with PBS before adding Optimem media with 2% Low IgG FCS and 5 mM Neu5Ac for three days, the media was collected on the third day (day 3), fresh media containing Neu5Ac was added and collected 2 days later (day 5), fresh media containing Neu5Ac was again added and collected on day 7. The individually collected media was centrifuged to remove cell debris and adjusted to 5 mM Tris-HCl pH8. The fusion protein was purified using Protein-A Sepharose. (C) An aliquot of the purified protein was hydrolyzed with 2M Acetic acid for 3 hrs at 80° C. to release the sialic acids followed by derivatization with DMB reagent and subjected to HPLC analysis. The area under each peak was obtained and the percent of Neu5Gc in each sample was determined relative to Neu5Ac. It can be seen that there was a marked reduction in Neu5Gc content relative to the control. (D) Total cell membranes from the same CHO cells were prepared and used for DMB-HPLC analysis. Again, it can be seen that there was a marked reduction in Neu5Gc content relative to the control. (E) CHO membrane proteins from the above experiments were separated by SDSPAGE and transferred onto nitrocellulose membranes. The expression of Neu5Gc was detected by incubating with polyclonal affinity purified chicken anti-Neu5Gc antibody. It can be seen that there was a marked reduction in Neu5Gc-expression on proteins, relative to the control.

FIG. 3 shows Neu5Ac content in urine from four human subjects fed with a food containing a large amount of glycosidically-bound Neu5Ac. It can be seen that there was a marked increase in Neu5Ac content of urine in the time period immediately following the ingestion of a Neu5Ac rich food. In analogy to the cellular experiments, this approach is expected to flood the human body with Neu5Ac. Repeated applications of this approach would eventually eliminate Neu5Gc from the human body.

### DETAILED DESCRIPTION

As used herein and in the appended claims, the singular forms “a,” “and,” and “the” include plural referents unless the context clearly dictates otherwise. Thus, for example, reference to “Neu5Gc” includes a plurality of such molecules and reference to “the Neu5Ac” includes reference to one or more Neu5Ac known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

Sialic acid (Sia) is a generic name for a family of acidic nine carbon sugars typically found as the outermost units of glycan chains on the vertebrate cellular glycocalyx and on secreted glycoproteins. Their location and widespread occurrence on all vertebrate cells allow them to be involved in processes such as ligand-receptor interactions, cell-cell recognition, cell-pathogen binding, inflammatory processes, immune responses and tumor metastases.

There are more than 50 kinds of Sias known in nature. Most are derived via biosynthetic modification of a Sia called N-acetylneuraminic acid (Neu5Ac). The addition of a single oxygen atom to the N-acetyl group of Neu5Ac gives rise to a very common variation called N-glycolylneuraminic acid (Neu5Gc). The surfaces of most primate cell types studied to date are dominated by these two major Sias.

In order for a Sialic (Sia) molecule to get attached to glycoconjugates, it must first be activated by conversion to the sugar nucleotide derivative, cytidine-monophosphate-Sia (CMP-Sia). Sias are converted to CMP-Sias in the nucleus, where it then return to the cytosol in order to be transported into the Golgi apparatus. The CMP-Sias serve as high-energy donors for attaching Sias to newly synthesized glycoconjugates on their way to the cell surface. The biosynthetic transformation of Neu5Ac to Neu5Gc occurs at this sugar nucleotide level, wherein the CMP-Neu5Ac hydroxylase (CMAH) catalyzes the transfer of an oxygen atom to CMP-Neu5Ac, generating CMP-Neu5Gc. CMP-Neu5Gc can then be transported into the Golgi apparatus and used, in the same manner as CMP-Neu5Ac, to add Neu5Gc to newly synthesized glycoconjugates. These two nucleotide sugars appear to be used interchangeably by the Golgi CMP-Sia transporter and by the mammalian sialyltransferases, which transfer Sia residues to cell surface and secreted glycoconjugates. Neu5Ac or Neu5Gc molecules that are released from glycoconjugates during lysosomal degradation processes can also be exported back into the cytosolic compartment by a specific transporter. There, the molecules are available as substrates for conversion to their respective CMP-Sia forms. Neu5Gc can be “recycled” for repeated use in Golgi sialation reactions.

The non-human sialic acid N-glycolyleneuraminic acid (Neu5Gc) contaminates both biotherapeutic products and the human body by becoming metabolically incorporated into cultured cells and into human tissues. In the first case the contamination arises from animal derived components in the culture medium and/or the animal cell lines used, and in the second case, from dietary intake. Preventing this Neu5Gc incorporation is very difficult because the extent of manipulation needed for either the culture medium or the diet is substantial.

Neu5Gc is perhaps the most widely expressed sialic acid in non-human mammalian cells. While humans are genetically deficient in producing Neu5Gc, small amounts are present in human cells. A dietary origin was suggested by human volunteer studies, and by observing that free Neu5Gc is metabolically incorporated into cultured human cells by unknown mechanisms. Research has shown that the incorporation of Neu5Gc may predominantly originate from dietary sources (Tangvoranuntakul, P. et al. Proc. Natl. Acad. Sci. (USA) (2003) 100:12045-12050.)

For example, red meat from sources such as beef, pork and lamb are particularly rich in Neu5Gc and are likely the primary sources of Neu5Gc in the human diet. Also, dairy products contain Neu5Gc, although at somewhat lower levels than in red meat.

Furthermore, biotherapeutics produced by genetically engineered mammalian cells or heterologous or xenogenic cells (e.g., transplants) can comprise Neu5Gc. The presence of Neu5Gc in human cells that are cultured in animal products for use in human therapeutic agents is also a potential source of antigenicity. More particularly, when human cells are cultured in serum from animals, they can take up and incorporate Neu5Gc, potentially resulting in immunological rejection if such cells are used for therapy (e.g., transplantation of human embryonic stem cell-derived grafts). For example, biotherapeutics produced by transformed mammalian cells (e.g., CHO cells) can produce either directly or through culture material containing Neu5Gc. Removal of Neu5Gc from such cultures is important.

It has been demonstrated that free Neu5Gc uptake occurs in a variety of mammalian cells and tissues, such as secretory cells, cancer cells, and blood vessels. Inhibitors of certain non-clathrin mediated (i.e. receptor independent) endocytic pathways reduce Neu5Gc accumulation. Studies with human mutant cells show that the lysosomal sialic acid transporter is required for metabolic incorporation of free Neu5Gc. Incorporation of glycosidically-bound Neu5Gc from exogenous glycoconjugates (relevant to human gut epithelial exposure to dietary Neu5Gc) requires the transporter, as well as the lysosomal sialidase, which presumably acts to release free Neu5Gc. Thus, exogenous Neu5Gc reaches lysosomes via pinocytic/endocytic pathways, and is exported in free form into the cytosol, becoming available for activation and transfer to glycoconjugates. In contrast, N-glycolylmannosamine (ManNGc) apparently traverses the plasma membrane inefficiently by passive diffusion and becomes available for conversion to Neu5Gc in the cytosol.

Most normal healthy humans have a certain amount of circulating anti-Neu5Gc antibodies, likely because of the fact that most humans ingest food sources or other material derived from non-human mammals containing high levels of Neu5Gc. For example, material that may inoculate a subject with a Neu5Gc include xenogenic (i.e., non-human) derived biological material (e.g., from culture methodologies such as stem cell cultures, bioreactor systems and the like) as well as implantation/transplantation material. Such material may promote an immunological response reducing the therapeutic success due to uptake and expression of Neu5Gc on the surface of any tissue developed from human cells exposed to Neu5Gc-containing products. This problem can also affect recombinant soluble biotherapeutic products.

Although Neu5Gc is a major Sia in most mammalian cells, it was long thought to be absent from healthy human tissues (Traving, C., et al., Cell. Mol. Life. Sci. 54: 1330-1349, 1998) Indeed, humans are genetically unable to synthesize Neu5Gc, due to an exon deletion/frame shift mutation in the human CMAH gene (Varki, A., Yearb. Phys. Anthropol. 44:54-69, 2002; Chou, H. H., et al. Proc. Natl. Acad. Sci. USA 95:11751-11756, 1998; and Irie, A., et al., J. Biol. Chem. 273: 15866-15871, 1998). It has been estimated that this mutation occurred in the hominid lineage—2.5 to 3 million years ago (Chou, H. H. et al., Proc. Natl. Acad. Sci. USA 99:11736-11741, 2002).

Despite the absence of any known alternative pathway for the synthesis of Neu5Gc in humans, various groups have used antibodies to study the expression of Neu5Gc in human tumors, particularly in various carcinomas (Hirabayashy, Y. et al., Jpn. J. Cancer Res. 78:251-260, 1987; Miyoshi, I. et al., Mol. Immunol. 23:631-638, 1986; Marquina, G. et al., Cancer Res. 56:5165-5171, 1996; Carr, A. et al., Hybridoma 19:241-247, 2000; Devine, P. L., et al., Cancer Res. 51:5826-5836, 1991; Kawachi S. et al., Int. Arch. Allergy Appl. Immunol. 85:381-383, 1988; and Higashi, H. et al., Jpn. J. Cancer Res. 79:952:956, 1998). Recent studies have re-explored these findings, confirming prior reports of Neu5Gc expression in human cancers and extending the finding to normal human tissues, including detecting small amounts of Neu5Gc in epithelial and endothelial cells of normal humans. Definitive confirmation resulted from releasing and purifying sialic acids from such tissues utilizing a fluorescent derivatized form of Neu5Gc by HPLC and mass spectrometry analysis (Tangvoranuntakal, P., et al., Proc. Natl. Acad. Sci. USA 100:12045-12050, 2003). It was shown that exogenously added free Neu5Gc is incorporated into cultured human carcinoma cells in vitro. In addition, oral ingestion studies of Neu5Gc in human volunteers provides evidence that the Neu5Gc found in human tissues can originate from dietary sources (Tangvoranuntakal, P., et al., Proc. Natl. Acad. Sci. USA 100:12045-12050, 2003), particularly from red meat and milk products.

Neu5Gc has been associated with cell proliferative disorders, cancer, tissue rejection and inflammation (“Neu5Gc associated disorders”). Because Neu5Gc is a non-human animal product dietary component, tissue transplants, biotherapeutic and cell-based therapies delivery such non-human Neu5Gc products to the body, where antibodies can react and cause inflammation, tissue rejection and the like.

Accordingly, the removal or reduction in Neu5Gc from humans and products consumed by humans can be used to reduce immunogenic responses and disease development. The disclosure provides methods for removal or reduction of existing Neu5Gc from a human subject, removal or reduction from an animal or animal product, removal or reduction from dietary consumables, and removal or reduction from therapeutics. In one embodiment, this method may be coupled with methods that reduce the uptake of Neu5Gc by an animal or human.

The disclosure demonstrates that incorporated Neu5Gc can be eliminated or substantially reduced by flooding the system with the human sialic acid N-acetylneuraminic acid (Neu5Ac), derivative, analogues or precursors thereof (e.g., ManNAc). Such methods can metabolically compete previously incorporated Neu5Gc or reduce or eliminate Neu5Gc in organisms or cells comprising a CMAH gene. The methods of the disclosure can be used to eliminate or reduce Neu5Gc either as it enters the cells for the first time or when it recycles from breakdown of preexisting cellular molecules. This method can be used in any mammalian system or organism including human cells and humans. For example, Neu5Ac is shown to be non-toxic to mice after prolonged exposure, accordingly this approach is applicable both to cells in culture, and to the intact mammalian body (e.g., human subjects).

In one embodiment, the disclosure provides a method to produce Neu5Gc-free biotherapeutics, cell products, cell cultures and products therefrom comprising flooding (e.g., flushing/chasing) a cell, culture or subject with N-acetylneuraminic acid (Neu5Ac), Neu5Ac precursor N-acetylmannosamine (ManNAc), or appropriate derivatives of either Neu5Ac or ManNAc. The amount of Neu5Ac, ManNAc or derivatives thereof comprises an amount sufficient to metabolically compete out Neu5Gc, either as it enters the cells for the first time and/or when it recycles from breakdown of preexisting cellular molecules. Thus, the method and compositions of the disclosure can be used to remove or reduce existing Neu5Gc in a subject, cell or animal, or can be used to prevent uptake or synthesis of Neu5Gc through competitive displacement.

As used herein a Neu5Ac composition generally refers to a composition comprising N-acetylneuraminic acid (Neu5Ac), Neu5Ac precursor N-acetylmannosamine (ManNAc), or derivatives of either Neu5Ac or ManNac. The N-acetylneuraminic acid (Neu5Ac), Neu5Ac precursor N-acetylmannosamine (ManNAc), or derivatives of either Neu5Ac or ManNac may be derived from natural sources or may be synthesized by standard chemical methods or may be generated by recombinant or enzymatic processes. The Neu5Ac used can also be bound in monomeric form to other molecules, or in a polymeric form (polysialic acid). In these cases, the bound Neu5Ac would be released by cellular processes and then act in a manner similar to the bound form.

The disclosure demonstrates that Neu5Gc may be removed or substantially decreased in such cells by flooding a cell, culture or subject with an N-acetylneuraminic acid (Neu5Ac), its precursor N-acetylmannosamine (ManNAc), or derivatives thereof including monomeric and polymeric forms of Neu5Ac. The amount of Neu5Ac, ManNAc or derivatives thereof comprise an amount sufficient to metabolically eliminate Neu5Gc, either as it enters the cells for the first time and/or when it recycles from breakdown of preexisting cellular molecules. In one embodiment, the method is used to replace previously incorporated Neu5Gc in a cell of subject. In yet another embodiment, the method is used to reduce the synthesis of Neu5Gc in cells or organisms that comprise a functional CMAH gene.

By “flooding” a cell or subject with Neu5Ac means providing a compositions high in Neu5Ac with little or no detectable Neu5Gc (e.g., at least 99%-100% free of Neu5Gc). It may be helpful to consider the process as flushing or chasing the Neu5Gc from the cell or organism. The amount of Neu5Ac used to flood a cell or subject is an amount sufficient to reduce and eliminate Neu5Gc, yet which is not toxic to the subject or cell. The amount will vary depending upon the size, weight, type of organism, culture conditions, amount of Neu5Gc in the subject or cell, whether the cell or organism has a functional CMAH gene or homolog and the like. The amount can be readily determined using skill in the art. The Neu5Ac used to flood a subject's system or a culture system may be a naturally occurring Neu5Ac (in free form or glycosidically-bound form) or a purely synthetic Neu5Ac or a combination thereof. Sources of Neu5Ac from dietary preparations are known and include, for example, Bird's nest soup. Methods of synthesizing Neu5Ac or derivatives thereof are known in the art.

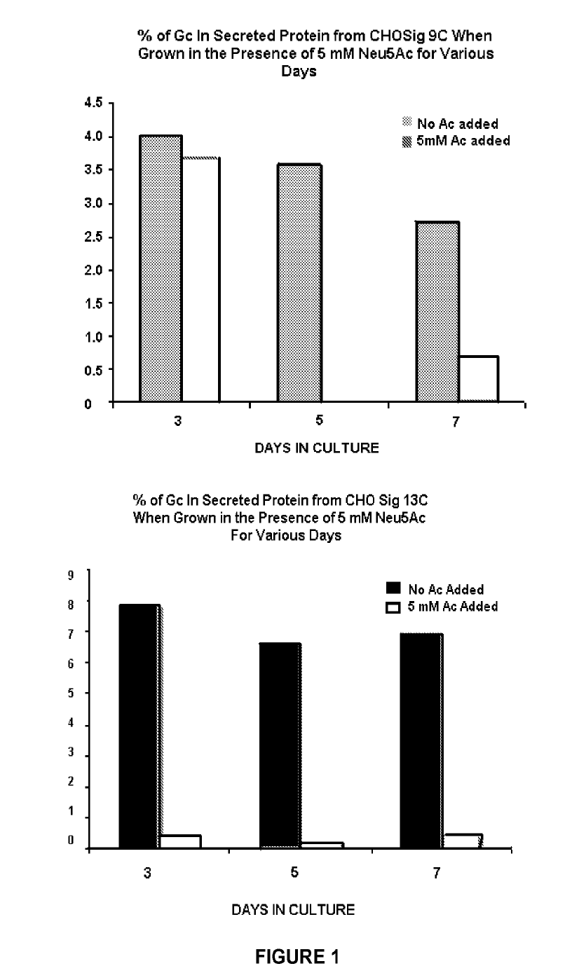

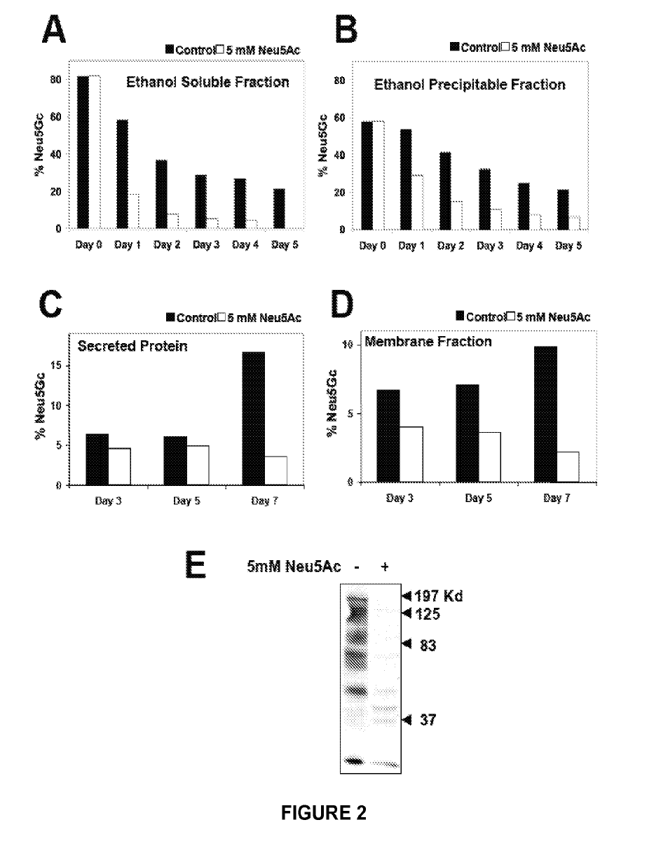

In one embodiment, a cell culture is grown and expanded or cultured under conditions to produce a desired product. The culture conditions can comprise a Neu5Gc-free media or a Neu5Gc-free media comprising N-acetylneuraminic acid (Neu5Ac), N-acetylmannosamine (ManNAc) or derivatives or analogues thereof in an amount sufficient eliminate pre-existing Neu5Gc in the cell or reduce production of Neu5Gc. For example, some cells currently used to produce biotherapeutic products e.g., CHO cells already contain and produce Neu5Gc and/or acquire Neu5Gc from the culture medium. In some embodiments, a cell line producing a biotherapeutic product of interest is cultured with an effective amount or increasing amounts of the Neu5Ac and/or precursors thereof or derivatives or analogues thereof to remove Neu5Gc from the culture. As demonstrated in FIG. 2E, the presence of Neu5Gc in the CHO cell membrane glycoproteins is greatly decreased by these increasing amounts of Neu5Ac, derivatives, analogues or precursor thereof. Likewise, the amount of Neu5Gc on a purified product secreted by the CHO cells steadily diminishes until it is undetectable (FIG. 1). Thus, the disclosure provides methods and compositions that can be used to eliminate Neu5Gc from existing biotherapeutic products that are already being produced in established animal cell lines, by addition of Neu5Ac to the medium.

Furthermore, the methods of the disclosure can effectively reduce the amount of Neu5Gc in organisms or cells derived there from that have an intact CMAH gene. The methods result in suppression of Neu5Gc production in such cells, in addition to the metabolic competition mechanism operating in human cells, which do not have an intact CMAH gene.

The terms “medium” or “media,” as used herein, refer to any culture medium used to grow and harvest cells and/or products expressed and/or secreted by such cells. Such “medium” or “media” include, but are not limited to, solution, solid, semi-solid, or rigid supports that may support or contain any host cell, including, by way of example, bacterial host cells, yeast host cells, insect host cells, plant host cells, eukaryotic host cells, mammalian host cells, CHO cells, prokaryotic host cells, E. coli, or Pseudomonas host cells, and cell contents. Such “medium” or “media” includes, but is not limited to, medium or media in which the host cell has been grown into which a polypeptide has been secreted, including medium either before or after a proliferation step. Such “medium” or “media” also includes, but is not limited to, buffers or reagents that contain host cell lysates (e.g., a polypeptide produced intracellularly and the host cells are lysed or disrupted to release the polypeptide).

In one embodiment, the cells or cell lines are grown in a first medium, washed and then grown in a second medium that is Neu5Gc-free and comprises Neu5Ac. In another embodiment, the medium comprises Neu5Ac at a concentration that is at least one-fold, two-fold, three-fold or higher than a Neu5Gc concentration in the medium.

Human Embryonic Stem Cells (hESCs) can potentially generate all body cell types, making them excellent candidates for cell and tissue replacement therapies. hESCs are typically cultured with animal-derived “serum replacements” on murine feeder layers. Both of these are sources of the non-human sialic acid Neu5Gc, against which many humans have circulating antibodies. Both hESC and derived embryoid bodies metabolically incorporate significant amounts of Neu5Gc under standard conditions (Martin, M. J., Muotri, A., Gage, F., and Varki, A., Human embryonic stem cells express an immunogenic non-human sialic acid. Nat. Med. 11:228-232, 2005). Using the methods of the disclosure the level of Neu5Gc can be reduced or eliminated by culturing the hESC, induced stem cells or embryoid bodies or cells derived therefrom under conditions of no or undetectable Neu5Gc and effective amounts of Neu5Ac (e.g., sufficient or increasing Neu5Ac concentrations) to replace Neu5Gc in the cell. Such medium will comprise Neu5Ac from about 0.1 mM to 20 mM or more. The method may include culturing the cells in medium that has been depleted of Neu5Gc by exposing the medium or components thereof to anti-Neu5Gc antibodies or using serum or media prepare or derived from transgenic organisms lacking a functional CMAH polypeptide/gene.

Serum or cells used in various compositions and methods of the disclosure may be derived from a CMAH transgenic animal. For example, feeder layer cells used in the culture of stem cell may be derived from a CMAH knockout transgenic animal. The method of the disclosure can be used in combination with such CMAH knockout cells or serum preparations to further “flush” any Neu5Gc from the culture systems that may have been incorporated during isolation, passaging or the like. A CMAH transgenic animal can have a knockout in a CMAH gene or a mutant CMAH gene. Such CMAH transgenic animals produce Neu5Gc-free products or products having a reduced Neu5Gc burden compared to wild-type animals. In another embodiment, serum replacements are now available to culture cells and tissues that lack animal products altogether.

Many general cell culture techniques can be used. Typical passaging, serum free media, serum containing media, cryopreservation and related techniques are well known in the art. The method for culturing will depend upon the organism and/or cell type and its growth requirements. In most instances, hESC's are cultured on animal feeder layers, most commonly mouse fibroblasts. However, for reasons discussed elsewhere herein, these feeder layers serve as additional sources for undesirable Neu5Gc, which can then be incorporated into the cells being cultured. Accordingly, in an attempt to be overcautious, it may be desirable to further remove any source of Neu5Gc by using human serum in the culture medium with human feeder layers, culturing the cells with effective concentrations of Neu5Ac to replace or compete out previously incorporated Neu5Gc or reduce production of Neu5Gc. The culture conditions can comprise Neu5Ac at about 0.1 mM to 20 mM or more.

As used herein, the term “devoid of Neu5Gc” or “free of Neu5Gc” intends that the ratio of Neu5Gc to total sialic acids is less than 5%, and even less than 1% or is undetectable. For example, the immunogenicity of Neu5Gc can be exploited by utilizing anti-Neu5Gc antibodies in an affinity column to remove any Neu5Gc present in the products. Alternatively, human cells of many varieties, tissues, embryoid bodies, neural lineage cells, carcinoma cells, skin cells, and organs (collectively referred to herein as “cells”) can be cultured with Neu5Ac and preserved in Neu5Ac-environments, so long as they are free of Neu5Gc. As discussed above, any Neu5Gc present in the culture or storage media can be incorporated into the cells, thus eliminating Neu5Gc and using an effective amount of Neu5Ac, one can eliminate Neu5Gc from such cells.

Besides hESCs, other cell types are within the scope of the disclosure including, for example, islet cells, endothelium, liver cells, kidney cells, cardiac cells, fibroblasts, etc. Most notably, however, progenitor cells and pluripotent cells that are not yet completely differentiated are of great use to scientific studies as potential sources for therapeutic treatments involving cellular implantation. In addition, the methods described herein can be used to preserve human organs prior to transplantation under conditions that avoid passing on anti-Neu5Gc antibodies or incorporation of additional Neu5Gc.

The methods of the disclosure may be used in combination with other agents that prevent incorporation of Neu5Gc. In one embodiment, the disclosure demonstrates the application of the methods of the disclosure to a human cell line or mouse cell line. Such cells are “chased” or “flushed” with medium containing increasing amounts of the human sialic acid Neu5Ac. The levels of Neu5Gc in the cells fall more rapidly when Neu5Ac is added to the medium with the effect peaking at about 5 mM. Thus, Neu5Ac can be used in a range of about 1 mM to more than 15 mM (e.g., 1, 2, 3, 4, 5, 6, 7, 8, 9, 10, 11, 12, 13, 14, 15 mM or more).

Cells currently used to produce biotherapeutic products, e.g., CHO cells, may already contain and produce Neu5Gc and/or acquire Neu5Gc from the medium. The methods of the disclosure reduce or remove Neu5Gc from the biotherapeutic product through a process, which can be generally described as metabolic competition during Neu5Gc recycling or Neu5Gc production. The method can be differentiated from a competitive “uptake” process wherein Neu5Gc uptake is competitively displaced by Neu5Ac. Rather, a method of the disclosure replaces previously incorporated Neu5Gc and/or synthesis of Neu5Gc for those cells comprising a functional CMAH gene or homolog. In one embodiment, the cell line is already producing a biotherapeutic product of interest and is then cultured with increasing amounts of the human sialic acid Neu5Ac and analogue or derivative thereof and/or precursors thereof to remove or reduce Neu5Gc. The Neu5Ac, derivative, analogue or precursor replaces (metabolically competes) with Neu5Gc previously incorporated into the cell culture. Over time the Neu5Gc is replaced or eliminated from culture. Typically the process reduces Neu5Gc a detectable amount compared to the same culture that does not receive any Neu5Ac. In some instances the reduction may be 1, 2, 3, 4, 5 fold or more.

The methods of the disclosure also provide methods of treating a human for Neu5Gc associated disorders or to prevent such disorders from occurring. The method comprises dosing (either by dietary consumption or medical intervention) the subject with Neu5Ac either as a large bolus, multiple deliveries or steadily increasing deliveries. The method delivers Neu5Ac as a metabolic competitor of Neu5Gc, which assists in removing Neu5Gc from the body. Typically the amount of Neu5Ac and Neu5Gc can be monitored by urine sample, tissue sample, blood, serum or plasma sampling.

The method comprises administering to the subject a composition comprising synthetic or naturally occurring free or bound Neu5Ac, Neu5Ac analogues, Neu5Ac derivatives, precursors or any combination thereof in an amount effective to reduce Neu5Gc content. Method of synthesizing Neu5Ac are known in the art. Neu5Ac can be obtained by enzymatic reaction, through recombinant organisms and through standard chemical reactions. Certain foods and bacterial polysaccharides also have a large content of Neu5Ac

In yet another embodiment, human urine is tested for Neu5Gc content after the individual has been on a Neu5Gc deficient diet for about three days (to clear out the gastrointestinal tract and eliminate any new sources of Neu5Gc). Following this period a 24-hour urine collection is used to determine the baseline level of Neu5Gc in the urine (e.g., being excreted from various cell types in the body and ending up in the urine). After doing such a measurement or plurality of measurement, the individual is then given a large dose or increasing larger doses of Neu5Ac (e.g., derived from natural sources). Repeated cycles of such Neu5Ac treatment can eventually result in gradual diminishing of the body burden of Neu5Gc.

Administration of Neu5Ac can be by any route (e.g., inravenous, oral, intraperitoneally and the like). Furthermore, the methods of the disclosure can utilize additional dietary methods including, but not limited to, (i) a diet of Neu5Gc-free foods, (ii) a diet of low amounts of Neu5Gc foods, (iii) a diet of high Neu5Ac foods or any combination of (i) or (ii) with (iii). The compositions and method are useful to eliminate incorporated Neu5Gc from a subject. In other words the compositions “flush” existing Neu5Gc from a subject of cell system. Although not wishing to be bound by an specific theory, as the Neu5Gc already incorporated into a subject or cell system is recycled, the excess Neu5Ac provided by the methods and compositions of the disclosure replace the Neu5Gc. This is in contrast to methods that compete with incorporation of Neu5Gc upon initial contact. However, it will be recognized that in addition to removing previously incorporated Neu5Gc, methods may be utilized to prevent uptake of Neu5Gc from a diet or cell culture system.

To prevent further uptake of Neu5Gc into the body from food sources, tablets or beverages containing Neu5Ac or ManNAc can be provided along with a meal that is known to contain Neu5Gc or along with a meal that comprises reduced-Neu5Gc or Neu5Gc-free foods/beverages. It is also possible to use certain foodstuffs that are known to be rich in sialic acids.

In one embodiment, a subject that presents with a Neu5Gc-associated disorder or is suspected of having a Neu5Gc associated disorder is administered a Neu5Ac composition, placed on a Neu5Ac diet, administered a Neu5Ac composition and placed on a low or Neu5Gc-free diet, or placed on a Neu5Ac diet and a low or Neu5Gc-free diet. As the Neu5Gc is metabolically replaced in the subject there will be a reduction in the Neu5Gc content of, for example, the urine.

The following examples are intended to illustrate but not limit the disclosure. While they are typical of those that might be used, other procedures known to those skilled in the art may alternatively be used.

### EXAMPLES

Experiments were performed with Chinese hamster ovary (CHO-Kl) cells and with epithelial cells isolated from a spontaneous tumor from a CMAH gene knock out mouse. However, since non-human cells often have large endogenous amounts of Neu5Gc, the consequences are more dramatic in human cells.

Free Neu5Ac and Neu5Gc are taken up and incorporated by the same pathways. Neu5Ac and Neu5Gc can be taken up by cells from an exogenous source and incorporated into endogenous glycoconjugates. Neu5Gc and Neu5Ac are used interchangeably by essentially all of the steps leading to their final incorporation into glycoconjugates.

By performing competition experiments in Caco-2 and human normal fibroblast cells, it was determined that Neu5Gc and Neu5Ac are taken up and incorporated via the same pathways. Both cell lines gave similar results. Feeding was done for 3 days with 3 mM Neu5Gc in the absence or presence (3 mM or 15 mM) of Neu5Ac in the media. Both molecules use the same pathways to enter into human cells and become available for metabolic incorporation. It is of course possible that there are minor differences in utilization of Neu5Gc and Neu5Ac by various enzymes and transporters in the pathways.

Feeding of human cells with free Neu5Ac can accelerate the elimination of pre-loaded Neu5Gc in whole cell membranes, and in secreted glycoproteins. Because of the transport and uptake of Neu5Gc from exogenous sources occurs, the disclosure provides methods and compositions to reduce such uptake or eliminate existing Neu5Gc from the cell or subject. As mentioned earlier, biotherapeutic products produced in human cell lines can be contaminated with Neu5Gc incorporated from animal-derived materials used in the culture medium. Furthermore, it will be very difficult for all of biopharma to change from the very well established and FDA-approved cell lines (e.g., CHO, BHK, murine myeloma) currently used for production.

293-T human kidney cells were grown in DME supplemented with 10% FCS. Cells were lifted from culture plate using 20 mM EDTA in PBS and allowed to grow to 50% confluence. At this point buffered 100 mM Neu5Gc was added to the culture in duplicate for a final 5 mM concentration, and the cells grown in this supplemented media for 3 days. At the end of this Neu5Gc pulse, the cells were once again lifted using 20 mM EDTA in PBS, pelleted, washed once with PBS to remove any excess Neu5Gc and then suspended in 30 ml of growth medium. Five ml of this cell suspension was added to each of 5 P-100 dishes. The last aliquot of cell suspension, time “0”, was immediately harvested by pelleting the cells, washing once with PBS, followed by suspending the cells in 1 ml of PBS and transferring to a 1.5 ml microcentrifuge tube. The cells were re-pelleted and frozen until all time points were collected. Buffered 100 mM Neu5Ac was added to each of the other 5 plates for the “Neu5Ac chase”, and an equivalent amount of media added to the “minus chase” samples. The cells were harvested at day 1, 2, 3, 4 and 5 by scraping into the culture media, collecting by pelleting, washing once with PBS, transferring to a 1.5 ml microcentrifuge tube, pelleting and freezing the cell pellet. At the end of the 5 days of chase, all collected cell pellets were homogenized in 300 μl of ice-cold 20 mM KPO4 pH 7 using 3-20 sec burst with a Fisher Sonicator. Glycoconjugate-bound sialic acids were precipitated by adding 700 μl of 100% ice-cold ethanol (final 70% ethanol) and incubation at −20° C. overnight. The samples were spun at 20000×g for 15 min and the supernatants transferred to a clean tube and dried on a speed vac. The precipitated glycoconjugates and dried ethanol supernatants were each suspended in 100 μl of 20 mM KPO₄ pH 7 with sonication. Sialic acids were released from both fractions by acid hydrolysis with 2M Acetic acid (final) and incubating at 80° C. for 3 hrs. Samples were passed through a Microcon-10 filter and the filtrate derivatized with DMB Reagent, for analysis of sialic acids by HPLC.

A similar approach was taken to CHO cells stably expressing a Siglec-Fc protein in the medium, except that the Neu5Gc pulse was omitted, and the secreted glycoprotein was captured on Protein-A Sepharose beads. The cells were also processed similarly, except that total cell membranes were pelleted by centrifugation. The sialic acid content of the secreted protein and cells membranes was determined by acid hydrolysis, DMB derivatization and HPLC. The cell membranes were also studied by Western-blotting with the chicken anti-Neu5Gc IgY.

Because Neu5Gc in cells is “recycled” in lysosomes, and reutilized for new synthesis of glycoconjugates, the disclosure provides a method of reducing such recycling and thereby eliminating Neu5Gc from the cell. The disclosure demonstrates that free Neu5Ac in the culture medium is take up by the cell and competes with preexisting Neu5Gc, preventing recycling of the latter. Also, the excess Neu5Ac would compete away any further incorporation of Neu5Gc from the medium. To study this possibility we pre-loaded human 293T cells with Neu5Gc, and then chased with medium in the presence or absence of 5 mM Neu5Ac. As shown in FIGS. 2A and 2B, the presence of Neu5Ac in the medium indeed resulted in more rapid clearance of ethanol-precipitable (glycosidically-bound) Neu5Gc from the cells and also from secreted glycoproteins. Thus, addition of Neu5Ac to the medium is a simple and non-toxic way to eliminate or reduce Neu5Gc contamination of human cells.

Most recombinant biotherapeutic glycoproteins are currently produced in non-human cells, the most popular of which are Chinese Hamster Ovary (CHO) cell lines. It is already known that biotherapeutic glycoproteins produced in CHO cells carry small amounts of Neu5Gc. Thus, the disclosure demonstrates a method of reducing Neu5Gc accumulation in cultured CHO cells. As shown in FIG. 2C-E, this was indeed successful, both for membrane glycoproteins and for a secreted recombinant protein. Given that the CHO cell already expresses its own endogenous Cmah enzyme, these data suggest that a novel mechanism is involved, in addition to competing out recycling Neu5Gc. Regardless of what that mechanism might be, this approach shows that reduction of Neu5Gc content of a recombinant glycoprotein can be achieved even in a non-human Cmah-positive cell line.

Pure chemically synthesized Neu5Ac is available in the needed amounts commercially. However, studies can use Neu5Ac derived from food sources.

As a prelude to treating humans, normal WT mice were fed with 1 mg/ml Neu5Ac in their drinking water for 18 weeks. These mice showed no signs of toxicity, by observation or complete blood counts and blood chemistry analysis, and final necropsy showed no histological abnormalities.

Human volunteers ingested bound Neu5Ac from edible bird's nest (EBN), a popular Chinese delicacy typically consumed as a soup and often referred to as the “Caviar of the East”. EBN originates from the saliva of the White-nest swiftlet (Aerodramus fuciphogus) inhabiting coastal regions of Vietnam, Thailand, Indonesia and the Philippines. EBN is an ideal source of dietary Neu5Ac since it is a natural food product that has very high Neu5Ac content (9% w/w). Moreover, it has been part of Chinese cuisine and traditional medicine for more than 1000 years. For preparation of bound Neu5Ac for ingestion, a typical traditional soup recipe was followed, in which the bird's nest was soaked overnight in water and then boiled in fresh water with broth and or sugar added for flavor. For preparation of free Neu5Ac for ingestion, the soup was made as above with the addition of vinegar in order to mildly acidify the broth and promote release of Neu5Ac. The vinegar was purchased from the grocery store. Such an extraction is similar to preparing a sweet and sour soup. The amount of free or bound Neu5Ac ingested by each individual was 1-3 g, which is similar to typical servings of EBN soup (see, e.g., www.chinesefood-recipes.com).

The Neu5Ac-containing sample was ingested by up to ten normal human volunteers who have avoided Neu5Gc containing products for 3 days, and only consumed fruit or vegetable juices (free of sialic acid) in the morning of the study. Avoidance of solid food is important because a full stomach could significantly delay the passage of the sample into the intestines, and the uptake of the test molecule. Following ingestion of the test sample, the subjects drank only fruit or vegetable juices at ˜4 ml/Kg/hour for the next six hours. All urine was collected starting 24 hours preceding ingestion and continued to be collected until 48 hours following ingestion (3 pools×24 hours=72 hours in total). Saliva samples was collected at 0, 6 and 24 and 48 hours after ingestion, after washing the mouth out thoroughly with saline, and chewing on a piece of parafilm for 1 minute. Blood samples (˜30 ml or ˜2 tablespoons) was collected at 0, 3 and 24 hours totaling ˜90 ml or 6 tablespoons. All samples were analyzed for free and bound Neu5Ac and Neu5Gc, as well as for N-acetylmannosamine (ManNAc) and N-glycolylmannosamine (ManNGc), the direct degradation products of these sialic acids. Aliquots of the samples were passed over Microcon filters with a molecular weight cutoff of 3000 daltons. For the bound sialic acids, the retentate was acid hydrolyzed and then again passed over similar Microcon-3 filters. The free sialic acids were then purified by Dowex anion exchange columns and these anionic sugars separated from neutral sugar fraction that will contain ManNAc and ManNGc. The latter were enzymatically converted back into sialic acids by the use of pyruvate lyase enzyme under addition of surplus sodium pyruvate to drive the reaction. Sialic acids were derivatized with DMB reagents (1,2-Diamino-4,5,Methylene-Dioxybenzene) in order to detect them by fluorescence after High Pressure Liquid Chromatography separation. Peaks of interest were directly analyzed by Mass Spectrometry for confirmation of chemical composition. For the purposes of quantitation, internal standard of other similar molecules were used, added to the urine sample before analysis.

The urine collected prior to ingestion provided information on baseline levels of urinary Neu5Ac and Neu5Gc excretion for the subject. During the next 24-hours of collection an increase of Neu5Ac excretion was observed due to gut absorption of the ingested food material, with a return to baseline in the 24-hour collection on day 3. The purpose of the blood sample collection is primarily to ensure that there are no significant changes in hematological and biochemical parameters. Note that these would be not highly unusual amounts for consumption on special occasions, amongst Chinese populations. Since each subject is his or her own control, the question is whether there is a significant decrease in urinary Neu5Gc excretion over time after repeated treatments. There are no prior data that might predict whether or not there might be differences in the results based on sex or ethnicity. Thus initial volunteer participants were investigators themselves, representing females and males of European, South Indian and Mexican origin (age range 31-56). All were currently in excellent health with no major medical problems. All non-essential medications, Neu5Gc-containing foods and any unusual foods were avoided for 72 hours prior to and 72 hours during the study.

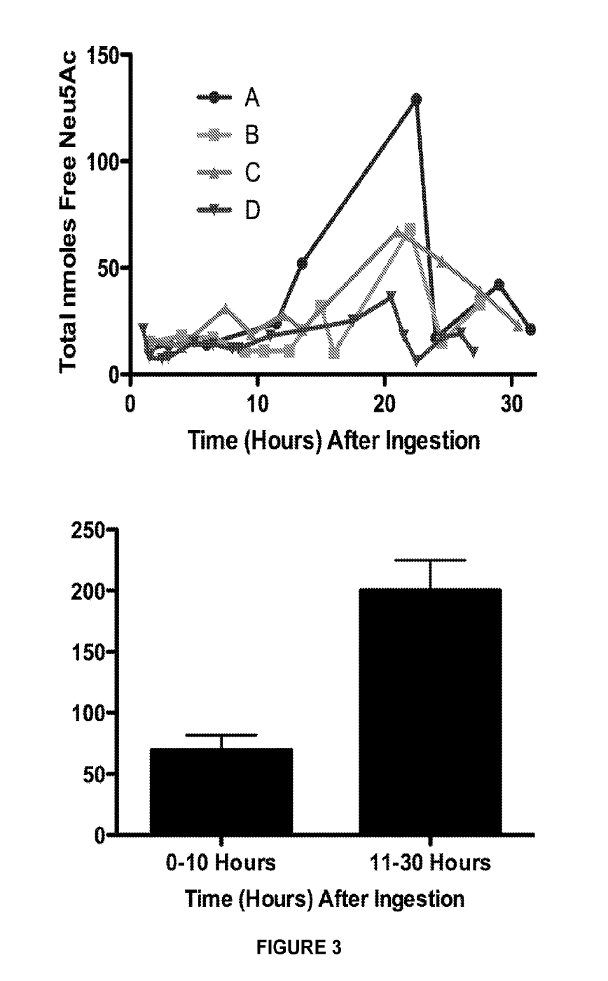

In one set of experiments four human subjects ingested bound ˜3G of Neu5Ac in edible bird's nest soup. All were in good health with no major medical problems. As shown in FIG. 3, urine collections (which were deliberately random, not timed) showed an increase in urinary excretion of Neu5Ac during the period 10-30 hours after ingestion. Later time points showed a return to baseline on day 2. No adverse effects were observed. After repeated treatments we expect to see the amount of baseline Neu5Gc in the urine fall.

A number of embodiments of the invention have been described. Nevertheless, it will be understood that various modifications may be made without departing from the spirit and scope of the invention. Accordingly, other embodiments are within the scope of the following claims.

What is claimed is:

1. A method comprising,
culturing a cell line comprising Neu5Gc with an effective amount or increasing amounts of free or glycosidically-bound N-acetylneuraminic acid (Neu5Ac), N-acetylmannosamine (ManNAc) or analogues or derivatives thereof for a sufficient period of time to remove or substantially reduce Neu5Gc present in a cell, cell line or a product produced by the cell or cell line.

2. The method of claim 1, wherein the cell line comprises a mammalian cell line used for therapeutic protein production.

3. The method of claim 1, wherein the cell line comprises a hESC, an induced stem cell or an embryoid body cell.

4. The method of claim 1, wherein the culturing with free or glycosidically-bound N-acetylneuraminic acid (Neu5Ac), N-acetylmannosamine (ManNAc) or analogues or derivatives thereof is performed after an initial culture with a Neu5Gc containing media component.

5. A method of reducing the amount of Neu5Gc in a biotherapeutic product comprising:
culturing a mammalian cell line comprising Neu5Gc under conditions for production of a biotherapeutic product in a medium comprising an effective amount or increasing amounts of free or glycosidically-bound N-acetylneuraminic acid (Neu5Ac), N-acetylmannosamine (ManNAc) or analogues or derivatives thereof.

6. The method of claim 5, wherein the cell line comprises a mammalian cell line used for therapeutic protein production.

7. The method of claim 5, wherein the cell line comprises a hESC, an induced stem cell or an embryoid body cell.

8. The method of claim 5, wherein the culturing with free or glycosidically-bound N-acetylneuraminic acid (Neu5Ac), N-acetylmannosamine (ManNAc) or analogues or derivatives thereof is performed after an initial culture with a Neu5Gc containing media component.

9. A method of preparing a tissue for transplantation comprising exposing the tissue to media lacking Neu5Gc and comprising an effective amount or increasing amounts of free or glycosidically-bound N-acetylneuraminic acid (Neu5Ac), N-acetylmannosamine (ManNAc) or analogues or derivatives thereof.

10. A method of treating an inflammatory disease or cancer comprising administering to a subject comprising Neu5Gc an effective amount or increasing amounts of free or glycosidically-bound N-acetylneuraminic acid (Neu5Ac), N-acetylmannosamine (ManNAc) or analogues or derivatives thereof until the level of Neu5Gc is reduced or removed from the subject.

11. The method of claim 10, wherein the disease or cancer is associated with Neu5Gc or anti-Neu5Gc antibodies.

12. A food, beverage or cell media product having elevated free or glycosidically-bound N-acetylneuraminic acid (Neu5Ac), N-acetylmannosamine (ManNAc) or analogues or derivatives thereof compared to the level of Neu5Gc for use in treating a Neu5Gc associated disease or disorder or reducing the Neu5Gc content in a subject's body.

13. The food, beverage or cell media of claim 12 comprising from about 0.1 mM to 20 mM or more of free or glycosidically-bound N-acetylneuraminic acid (Neu5Ac), N-acetylmannosamine (ManNAc) or analogues or derivatives thereof.

14. A method of treating a subject with a Neu5Gc associated disease or disorder comprising reducing or eliminating Neu5Gc dietary consumption and administering an effective amount or increasing amounts of free or glycosidically-bound N-acetylneuraminic acid (Neu5Ac), N-acetylmannosamine (ManNAc) or analogues or derivatives thereof to remove pre-existing Neu5Gc from the subject.

15. The method of claim 14, wherein the free or glycosidically-bound N-acetylneuraminic acid (Neu5Ac), N-acetylmannosamine (ManNAc) or analogues or derivatives thereof is from a dietary source.

16. The method of claim 14, wherein the free or glycosidically-bound N-acetylneuraminic acid (Neu5Ac), N-acetylmannosamine (ManNAc) or analogues or derivatives thereof is from a synthetic source.

17. A method of eliminating Neu5Gc from a biotherapeutic product or a cell line used for production of a biotherapeutic product comprising culturing the cells in a Neu5Gc-free media comprising an effective amount or increasing amounts of free or glycosidically-bound N-acetylneuraminic acid (Neu5Ac), N-acetylmannosamine (ManNAc) or analogues or derivatives thereof to remove previously incorporated Neu5Gc from the cell line.

18. A method of reducing production of Neu5Gc in an animal comprising a Neu5Gc enzymatic pathway, the method comprising administering to the animal an effective amount of Neu5Ac, derivative, analogue, or precursor wherein the effective amount reduces or eliminates that production of Neu5Gc in the animal.
